(19) United States
(12) Patent Application Publication
(10) Pub. No.: US 2003/0104490 A1
(43) Pub. Date: Jun. 5, 2003

(54) Assay

(76) Inventors: Laura Margaret Fletcher, Brislington (GB); Jeremy Myles Tavare, Redland (GB)

Correspondence Address: Daniel A Scola Jr, Hoffmann & Baron, 6900 Jericho Turnpike, Syosset, NY, 11791, US

(21) Appl. No.: 10/311,409

(22) PCT Filed: Jun. 18, 2001
(86) PCT No.: PCT/GB01/02683

(30) Foreign Application Priority Data
Jun. 16, 2000 (GB) 0014838.7

(51) Int. Cl. G01N 33/53; G01N 33/567; C07H 21/04; C07K 14/65

(52) U.S. Cl. 435/7.2; 530/324; 536/23.5

(57) ABSTRACT

There is disclosed a method of determining whether a candidate compound mimics or antagonizes effects of insulin, comprising: (a) providing a transfected host cell comprising a nucleic acid sequence which encodes a protein construct comprising an IRAP protein moiety and a detectable protein moiety, wherein the detectable protein moiety is fused to a luminal domain of the IRAP protein moiety and further wherein the detectable protein moiety is capable of being assayed by a first detection method capable of detecting the protein moiety when located exofacially but not when located intracellularly and a second detection method capable of detecting the protein moiety both when located exofacially and when located intracellularly; (b) contacting the transfected host cell with a candidate compound; (c) measuring the amount of the exofacial protein moiety by the said first detection method and measuring the total amount of exofacial and intracellular detectable protein moiety by the said second detection method; and (d) comparing the amount of exofacial detectable protein moiety with the total amount of exofacial and intracellular detectable protein moiety to provide a measure of the extent of GLUT4 vesicle translocation. In an alternative method, the protein construct comprises an IRAP protein moiety, a first detectable protein moiety which is fused to a luminal domain of the IRAP protein moiety and a second detectable protein moiety which is fused to a cytoplasmic domain of the IRAP protein moiety, wherein the first detectable protein moiety is capable of being assayed by a first detection method capable of detecting the first protein moiety when located exofacially but not when located intracellularly and wherein the second detectable protein moiety is capable of being assayed by a second detection method capable of detecting the second protein moiety when located intracellularly. The assays of the invention may be used to determine whether a “candidate compound” is a mimic or antagonist of insulin. The present invention may be used to test libraries of compounds in an automated high throughput screen.

This invention relates to a method of determining whether a candidate compound mimics or antagonizes effects of insulin. In particular, in accordance with the present invention, the level of GLUT4 vesicle translocation can be quantified after insulin stimulation of host cells which express a chimeric IRAP protein. The method of the invention may, in particular, be used in a high-throughput screening method in the search for drugs that may potentially influence the action of insulin on-glucose uptake.

### BACKGROUND OF THE INVENTION

Insulin stimulated glucose uptake is the major physiologically relevant effect of insulin. If a compound could be discovered that specifically increased glucose uptake into cells this would have considerable benefit for the treatment of both type I and type II diabetes.

Two glucose transporters exist (GLUT1 and GLUT4) in insulin responsive fat and muscle cells. Insulin stimulates glucose uptake into muscle and fat cells by promoting the translocation of these transporters from an intracellular sequestered localisation to the plasma membrane. The most important effect in terms of net glucose uptake is the translocation of GLUT4. Furthermore, as GLUT1 is ubiquitously expressed (GLUT4 is restricted to muscle and adipose cells) then any effective treatment of diabetes must necessarily be specific for promoting the translocation of GLUT4.

Traditional assays of glucose uptake using radioactive glucose, or ligand binding assays such as tritiated cytochalasin B binding or the binding of tritiated or biotinylated bis-mannose labels (which directly bind to cell surface glucose transporters), do not distinguish an effect of insulin on either GLUT1 or GLUT4.

WO 95/09240 discloses a chimeric GLUT transporter including a GLUT transporter polypeptide fused to a detectable heterologous polypeptide. The chimeric GLUT transporter disclosed may be used in a method of determining whether a candidate compound mimics or antagonizes effects of insulin.

Insulin-responsive aminopeptidase (IRAP) is a component of GLUT4-containing vesicles and is involved in the insulin-signalling pathway. IRAP and GLUT4 completely co-localise. Reference is made to WO96/09317 which refers to this aminopeptidase as “GTVap”.

Thurmond et al., J. Biol. Chem. 273, 33876-33883 (1998), describe an experiment in which increased expression of Munc18c inhibits insulin-stimulated translocation of eGFP-IRAP/vp165 in 3T3L1 adipocytes.

### SUMMARY OF THE INVENTION

According to a first aspect of the present invention, there is provided a method of determining whether a candidate compound mimics or antagonizes effects of insulin, comprising:

(a) providing a transfected host cell comprising a nucleic acid sequence which encodes a protein construct comprising an IRAP protein moiety and a detectable protein moiety, wherein the detectable protein moiety is fused to a luminal domain of the IRAP protein moiety and further wherein the detectable protein moiety is capable of being assayed by a first detection method capable of detecting the protein moiety when located exofacially but not when located intracellularly and a second detection method capable of detecting the protein moiety both when located exofacially and when located intracellularly;

(b) contacting the transfected host cell with a candidate compound;

(c) measuring the amount of the exofacial protein moiety by the said first detection method and measuring the total amount of exofacial and intracellular detectable protein moiety by the said second detection method; and

(d) comparing the amount of exofacial detectable protein moiety with the total amount of exofacial and intracellular detectable protein moiety to provide a measure of the extent of GLUT4 vesicle translocation.

According to a second aspect of the present invention, there is provided a method of determining whether a candidate compound mimics or antagonizes effects of insulin, comprising:

(a) providing a transfected host cell comprising a nucleic acid sequence which encodes a protein construct comprising an IRAP protein moiety, a first detectable protein moiety which is fused to a luminal domain of the IRAP protein moiety and a second detectable protein moiety which is fused to a cytoplasmic domain of the IRAP protein moiety, wherein the first detectable protein moiety is capable of being assayed by a first detection method capable of detecting the first protein moiety when located exofacially but not when located intracellularly and wherein the second detectable protein moiety is capable of being assayed by a second detection method capable of detecting the second protein moiety when located intracellularly;

(b) contacting the transfected host cell with a candidate compound;

(c) measuring the amount of the first protein moiety by the said first detection method representing the amount of the first protein moiety located exofacially and measuring the amount of the second detectable protein moiety by the said second detection method representing the total amount of the second detectable protein moiety; and

(d) comparing the amount of exofacial first detectable protein moiety with the total amount of the second detectable protein moiety to provide a measure of the extent of GLUT4 vesicle translocation.

The measurements in step (c) of each of the first and second aspects of the invention may be carried out simultaneously or sequentially. Where they are carried out sequentially, the order in which they are carried out is unimportant.

As used herein, the expression “located exofacially” means positioned on the extracellular face of the plasma membrane. The expression “located intracellularly” refers to the relevant protein moiety being located within the cell which includes being located on the cytosolic face of the plasma membrane as well as within vesicles within the cell itself.

The assays of the first and second aspects of the invention may be used to determine whether a “candidate compound” (which may include a mixture of compounds), for example as may be found in a compound library, is a mimic or antagonist of insulin. The present invention may be used to test libraries of compounds in an automated high throughput screen. The assays of the invention are of most interest in identifying candidate compounds which are antagonists of insulin; such compounds are of major pharmaceutical interest.

Where the method of the invention is used to identify an antagonist of insulin, the method will also include a step in which insulin is added. Insulin may be added either before or after the transfected host cell is contacted with the candidate compound. Preferably, insulin is added after the transfected host cell is contacted with the candidate compound.

Where the method of the invention is used to identify an insulin mimic, insulin is not introduced into the test system.

According to a third aspect of the invention, there is provided a chimeric IRAP protein comprising an IRAP protein moiety and a detectable protein moiety, wherein the detectable protein moiety is fused to a luminal domain of the IRAP protein moiety and further wherein the detectable protein moiety is capable of being assayed by a first detection method capable of detecting the protein moiety when located exofacially in a cell but not when located intracellularly and a second detection method capable of detecting the protein moiety both when located exofacially in a cell and when located intracellularly.

According to a fourth aspect of the invention, there is provided a chimeric IRAP protein comprising (a) an IRAP protein moiety, (b) a first detectable protein moiety which is fused to a luminal domain of the IRAP protein moiety, said first detectable protein moiety being capable of being assayed by a first detection method capable of detecting the protein moiety when located exofacially in a cell but not when located intracellularly, and (c) a second detectable protein moiety which is fused to a cytoplasmic domain of the IRAP protein moiety, said second detectable protein moiety being capable of being assayed by a second detection method capable of detecting the protein moiety when located intracellularly.

According to a fifth aspect of the invention, there is provided a DNA molecule which encodes the chimeric IRAP protein of the third or fourth aspect of the invention.

### BRIEF DESCRIPTION OF THE DRAWING

FIG. 1 displays representative images demonstrating insulin-induced translocation of IRAP-GFP in CHO-T cells visualised using anti-GFP antibodies; and

FIG. 2 shows the increase in binding of anti-GFP antibodies to surface of unpermeabilised CHO-T cells expressing an IRAP-GFP construct after insulin treatment.

### DETAILED DESCRIPTION OF THE INVENTION

The method of the first aspect of the invention, in general terms, consists of transfecting insulin-sensitive cells with an IRAP construct which encodes a chimeric IRAP polypeptide containing a detectable protein moiety fused to a luminal domain of the IRAP protein moiety. The detectable protein moiety is detectable by a first detection method which is capable of detecting the protein moiety when it has been translocated from its starting location in intracellular GLUT4 vesicles to the cell surface membrane where the detectable protein moiety will lie exofacially (that is to say positioned extracellularly), but which method is not capable of detecting the detectable protein moiety when it has not been so translocated and hence lies intracellularly. The detectable protein moiety is also detectable by a second detection method which is capable of detecting the protein moiety both when it has been translocated to an exofacial location and also when it has not been so translocated and hence lies intracellularly.

Thus, when a transfected host cell expresses the chimeric IRAP polypeptide, the effect of insulin on GLUT4 translocation can be quantified in an assay by comparing the amount of the detectable protein moiety detected using the first detection method with the amount of detectable protein moiety which is detected by the second detection method. An advantage of the double detection approach employed in the present invention is that it allows one to correct for variations in IRAP expression level, and so adds an extra level of confidence to the assay. The invention can be used to screen for drugs which mimic or antagonize the effect of insulin.

The method of the second aspect of the invention is comparable with the first, with the exception that the chimeric IRAP protein includes two detectable protein moieties, one fused to the luminal domain and one fused to the cytoplasmic domain of the IRAP protein moiety.

The first detection method is preferably an immunological detection method in which an antibody epitope on the detectable protein moiety is detected using a labelled antibody having a high affinity for the epitope. Alternatively, the detectable protein moiety may be detected using a first antibody having a high affinity for the epitope tag and a second fluorescently labelled antibody having a high affinity for the first antibody. Such immunological assays will only detect the detectable protein moiety when located exofacially. Immunological assays of this type are well known in the art. Suitable labelled antibodies may be made by procedures known in the art.

The epitope on the detectable protein moiety which is to be detected by the first detection method may be one which is a natural epitope of the detectable protein moiety; thus, where the detectable protein moiety is a GFP protein moiety, this will contain a number of epitopes to which a specific antibody may be raised. As an alternative, the detectable protein moiety may be engineered to include a suitable epitope tag, such as a haemagglutinin (HA), c-myc, FLAG, Glu-Glu or His₆ epitope tag.

The second detection method is preferably one which relies on the generation of a characteristic emission spectrum, which is distinguishable from that which may be obtained using the first detection method, which can then be analysed by a suitable device such as a spectrophotometer. For example, the detectable protein moiety may comprise a fluorescent protein moiety, such as a green fluorescent protein (GFP) or the like. A fluorescent protein as used herein refers to a protein capable of fluorescence when excited with appropriate electromagnetic radiation and includes fluorescent proteins whose amino acid sequences are either natural or engineered. Such fluorescent protein moieties generate a characteristic emission spectrum in response to excitation at a particular wavelength and so will inherently assay the total amount of the protein moiety in the system under analysis.

A green fluorescent protein, as used herein, is a protein that fluoresces green light. A blue fluorescent protein (which may also be used in the invention) is a protein that fluoresces blue light. GFPs have been isolated from the Pacific Northwest jellyfish, Aequorea victoria, the sea pansy, Renilla reniformis, and Phialidium gregarium. W. W. Ward et al., Photochem. Photobiol., 35:803-808 (1982); L. D. Levine et al., Comp. Biochem. Physiol., 72B:77-85 (1982).

A variety of Aequorea-related GFPs having useful excitation and emission spectra have been engineered by modifying the amino acid sequence of a naturally occurring GFP from Aequorea victoria. (D. C. Prasher et al., Gene, 111:229-233 (1992); R. Heim et al., Proc. Natl. Acad. Sci., USA, 91:12501-04 (1994)).

Another fluorescent protein which may be used in the invention is red fluorescent protein isolated from the IndoPacific sea anemone relative Discosoma striata and available from Clontech Laboratories, Inc., Palo Alto, Calif., USA.

Other suitable detectable protein moieties are those which rely on a substrate for generation of a signal. For example, the enzyme luciferase when contacted with its substrate luciferin generates a characteristic emission spectrum. Another example is a detectable protein moiety based on β-galactosidase which, when contacted with its substrate, yields a characteristic emission. In both of these examples it is necessary to select a protein moiety/substrate combination which allows the total amount of the protein moiety to be assayed. More particularly, this means that the substrate must be one which is membrane permeable in order that the substrate may diffuse into cells to enable the necessary reaction between substrate and protein moiety to take place.

It is presently preferred that the detectable protein moiety of the method of the first aspect of the invention is a fluorescent protein moiety such as a green fluorescent protein which replaces the amino peptidase domain of IRAP in the chimeric protein. This may be assayed immunologically by way of a labelled antibody specific for an epitope of the fluorescent protein moiety, and fluorometrically using a standard fluorimeter.

In the case of the method of the second aspect of the invention, it is presently preferred that the first detectable protein moiety is a protein moiety including an epitope tag as described above and the second detectable protein moiety is a fluorescent protein moiety such as a green fluorescent protein which replaces the amino peptidase domain of IRAP in the chimeric protein.

The nucleic acid construct coding for the chimeric IRAP protein may be made by standard techniques of molecular biology. The DNA sequence of the IRAP protein encodes a protein which contains a cytoplasmic domain (amino acid residues 1 to 109), a trans-membrane portion (amino acid residues 110 to 131) and a luminal portion (amino acid residues 132-916 of which residues 132-154 represent a “stalk” and residues 155-916 the amino peptidase catalytic domain). DNA sequences encoding part or all of the IRAP protein may be engineered by known methods to include a nucleic acid sequence encoding the detectable protein moiety (or detectable protein moieties) such that the protein product expressed by the construct comprises the IRAP protein moiety fused to the detectable protein moiety (or detectable protein moieties). Thus, for example, DNA sequences coding for functional fluorescent protein moieties are available commercially and may be used to synthesise nucleic acid constructs coding for the chimeric IRAP protein of the invention. Additionally, construction of suitable vectors containing the desired coding and control sequences employs standard techniques that are well understood in the art.

In the DNA construct, a DNA sequence coding for the detectable protein moiety is joined at the end of the DNA sequence coding for the luminal portion of the IRAP protein (in other words, the whole of the DNA sequence coding for the luminal portion of the IRAP protein remains intact) or replaces all or a part of the DNA sequence coding for the luminal portion of the IRAP protein.

In a preferred embodiment of the invention, a DNA sequence encoding an incomplete IRAP protein missing its catalytic amino peptidase domain is used to synthesise a suitable DNA construct coding for the chimeric IRAP protein; thus, in the resultant DNA construct, the sequence coding for the amino peptidase domain of IRAP is replaced by a sequence coding for the detectable protein moiety. Experimental results demonstrate that the protein expressed by such a DNA construct traffics and responds to insulin in a manner indistinguishable from endogenous IRAP and GLUT4 itself.

A host cell is transfected with a suitable vector including the DNA encoding the chimaeric IRAP construct by methods known in the art. Nucleic acids used to transfect cells with sequences coding for expression of the chimeric IRAP protein generally will be in the form of an expression vector including expression control sequences operatively linked to a nucleotide sequence coding for expression of the polypeptide. As used, the term “nucleotide sequence coding for expression of” a polypeptide refers to a sequence that, upon transcription and translation of mRNA, produces the polypeptide. This can include sequences containing, e.g., introns. As used herein, the term “expression control sequences” refers to nucleic acid sequences that regulate the expression of a nucleic acid sequence to which it is operatively linked. Expression control sequences are operatively linked to a nucleic acid sequence which the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, maintenance of the correct reading frame of that gene to permit proper translation of the mRNA, and stop codons. Since long term expression of the chimeric IRAP protein may be toxic to cells, it is convenient if the expression of the or each construct is under the control of an inducible promoter, such as tetracycline responsive promoters (e.g. the Clontech T-Rex system), Ecdysone-based systems and the well known metallothionein promoter.

The resultant transfected host cell expresses the chimeric IRAP protein, preferably under the control of an inducible promoter. Suitable host cells which may be transfected to produce a stable cell line include adipocytes, for example 3T3-L1 adipocytes, skeletal muscle cells or any other insulin-responsive cell.

The transfected host cells expressing the chimeric IRAP protein may be used in an assay for determining whether a candidate compound mimics or antagonizes the effects of insulin.

In an assay for determining whether a candidate compound mimics the effects of insulin, the assay in accordance with the first or second aspect of the invention is carried out in the absence of insulin and the extent of GLUT4 vesicle translocation is compared with that which occurs in a control experiment from which the candidate compound (and insulin) is absent. A degree of GLUT4 vesicle translocation greater than the control indicates a compound which is a mimic of insulin.

In an assay for determining whether a candidate compound antagonizes the effects of insulin, the assay in accordance with the first aspect or the second aspect of the invention is carried out in the presence of insulin and the extent of GLUT4 vesicle translocation is compared with that which occurs in a control experiment in which the candidate compound is absent (but in the presence of the same concentration of insulin). A degree of GLUT4 vesicle translocation less than the control indicates a compound which is antagonizes the effects of insulin.

In more detail, the assay of the invention may be carried out as follows. A culture of the stable transfected cells is incubated in the presence of a candidate compound under suitable conditions in the presence of a suitable growth medium, with or without insulin (depending on whether the assay is being carried out to identify an insulin mimic or antagonist), for a known period of time. For example, cells may be incubated with the candidate compound for a suitable length of time (e.g. 15 min-3 hr) in a growth medium such as Dulbecco's Modified Eagles Medium (DMEM) or HAMS F12 medium at 37° C. in a humidified incubator containing 5% CO₂. Alternatively, the cells would be maintained at 37° C. in air and the medium supplemented by HEPES and sodium bicarbonate in order to maintain a suitable pH. In all cases, the cells would be in the absence of added serum for the incubation time. If insulin is subsequently added, this may for example be for a period of from 5 minutes up to an hour.

Where expression of the chimeric IRAP protein is under the control of an inducible promoter, the IRAP-GFP fusion protein will be induced by addition of inducer to the cells and the incubation carried out for a known period of time from addition of the inducer.

After the incubation period, the cells are analysed for the presence of the chimeric IRAP protein present at the cell surface using the first detection method. The total level of the chimeric IRAP fusion protein expressed is then determined using the second detection method. The result, typically a signal intensity, obtained from the first detection method will be divided by the result, again typically a signal intensity, obtained from the second detection method to provide a ratio. This ratio provides an additional level of confidence in the data generated. For example, calculating such a ratio allows one to correct for any variabilities in the number of transfected cells in the field of view, the level of induction of expression of the fusion protein by inducer, and the health of the cells (a decrease in signal will be observed if cells die or drop off the surface of the well). A major result of this additional correction is a decrease in the frequency of false positives.

The assay of the invention is suitable for use as a high throughput screening assay in which parallel assays may be automated on standard multiwell plates.

The methods of the first and second aspects of the invention may also be used to determine whether a candidate compound mimics or antagonizes the effect of a stimulus other than insulin which causes GLUT4 vesicle translocation. Other stimuli which can cause GLUT4 vesicle translocation are osmotic stress (for example which may be caused by sorbitol), concentrations of non-hydrolysable GTP analogues (e.g. GTPγS), AMP kinase activators (e.g. 5-amino4-imidazolecarboxamide riboside; AICAR) and tyrosine phosphatase inhibitors (e.g. vanadate). In this alternative assay, the cells would be contacted with an alternative stimulant, for example sorbitol rather than insulin, after incubation with the candidate compound. Otherwise, the methodology of the assay is the same as for the assay for identifying antagonists of insulin.

The invention will now be illustrated by reference to the following examples.

### EXAMPLE 1

Detection of Insulin-Induced Translocation of GLUT4-Containing Vesicles to the Plasma Membrane Using Immunofluorescence

The IRAP-GFP construct used in this example was constructed using an IRAP fragment 66-533 (from the start site to the end of the extracellular ‘stalk’ region) which was generated by PCR using specific primers from a partial cDNA IRAP clone available to the inventors. The resulting fragment was cloned into the pcDNA3 vector using the flanking restriction enzyme sites. GFP3 with a C-terminal myc-tag was also generated by PCR using specific primers and the product cloned into pcDNA3 downstream of the IRAP insert.

CHO-T cells were transfected or microinjected with the IRAP-GFP construct and 24 h later were stimulated without or with 200 nM insulin for 30 min. This was preceded by treatment with an inhibitor (e.g. wortmannin) of insulin signalling.

Subsequently cells were fixed and stained with anti-GFP antibodies (Roche) without prior permeabilisation, as described below, in order to visualise GFP exposed on the surface of the plasma membrane only.

Immunofluorescence of Cells

After washing twice in PBS at room temperature, cells were fixed using 4% (w/v) paraformaldehyde for 20 minutes at room temperature. For staining, fixed cells were incubated in the appropriate antibodies (in this case anti-GFP monoclonal antibodies) diluted in PBS containing 3% (w/v) BSA for 30 min to 1 h followed by incubation in a 1:200 dilution of the TRITC-conjugated secondary antibody for 30 min to 1 h. All antibody incubation steps were followed by extensive washes in PBS.

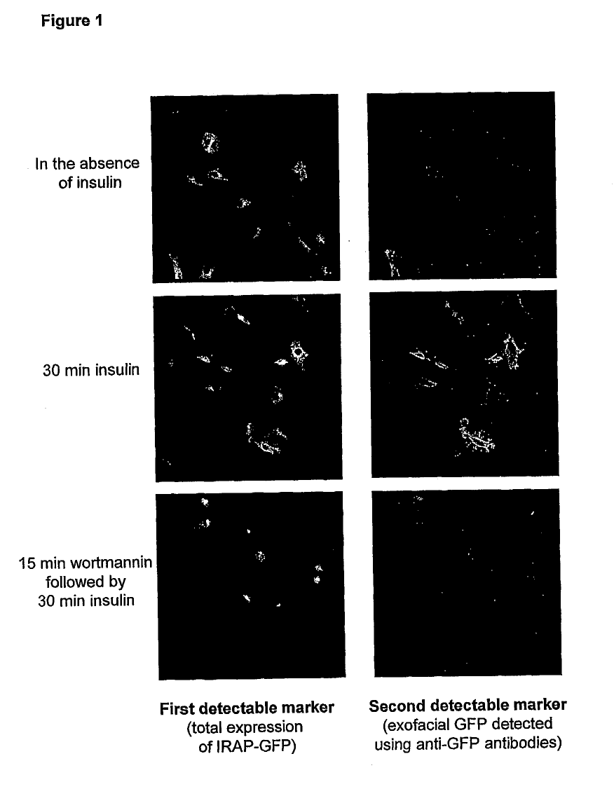

The total IRAP-GFP expression level is given by the level of GFP fluorescence (and this corresponds to the second detection method of the first aspect of the invention). The IRAP-GFP exposed at the cell surface is given by the level of staining with the anti-GFP antibodies (and this corresponds to the first detection method of the first aspect of the invention). FIG. 1 shows examples of the images that may be obtained by each detection method.

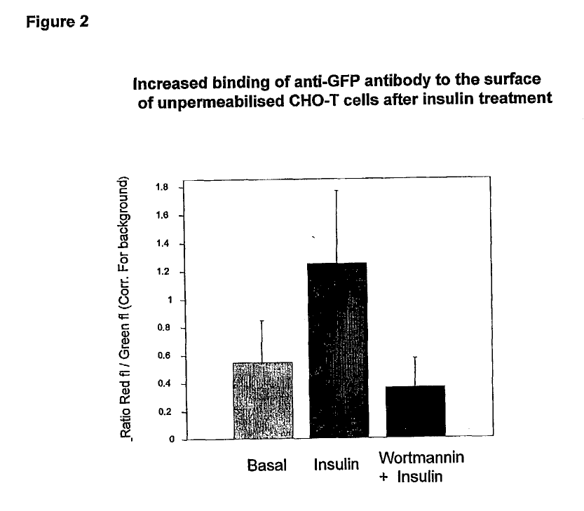

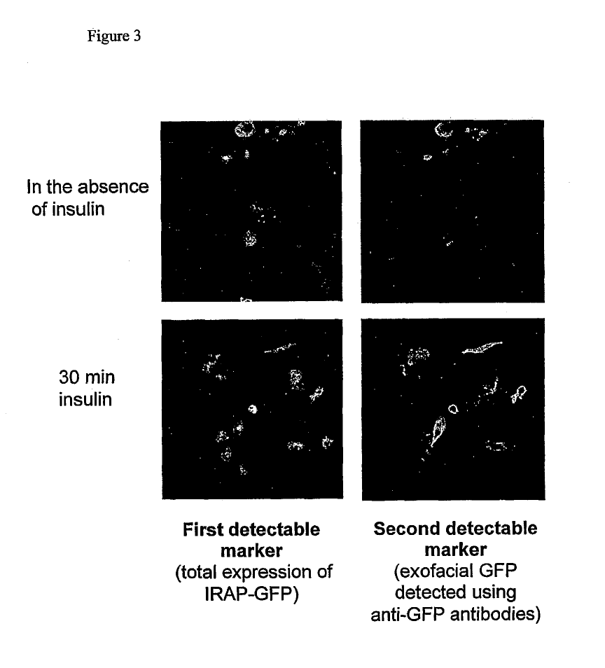

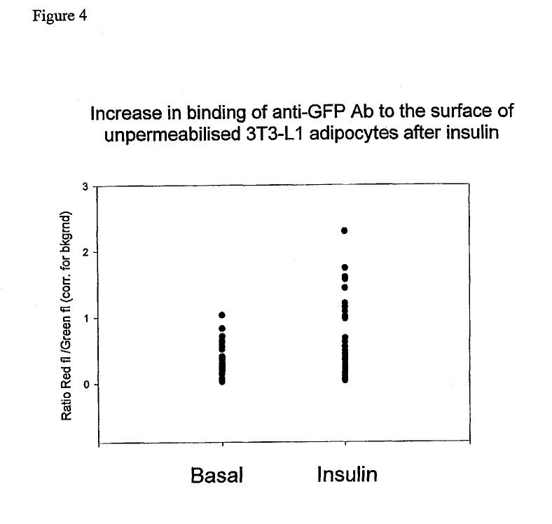

Fluorescence intensity due to IRAP-GFP fluorescence and surface IRAP-GFP immunostaining was then computed from cells imaged in 20 fields of view for each treatment condition. Results are expressed as an average ratio of surface:total IRAP-GFP. The results obtained are shown in Table 1 and FIG. 2.

TABLE 1

| TREATMENT | MEAN R/G | SD |
|---|---|---|
| Basal | 0.546 | 0.302 |
| Insulin | 1.252 | 0.514 |
| Wortmannin | 0.363 | 0.208 |

### EXAMPLE 2

Detection of Insulin-Induced Translocation of GLUT4-Containing Vesicles to the Plasma Membrane in 3T3-L1 Adipocytes.

Differentiation of 3T3-L1 fibroblasts into the adipocyte phenotype was performed as follows:

3T3-L1 fibroblasts (between passages 4 and 12) were seeded onto 22 mm glass coverslips and allowed to grow to confluence. Differentiation into adipocytes was induced in DMEM containing 10% (v/v) myoclone-plus FCS, 0.25 μM dexamethasone, 0.5 mM isobutylmethylxanthine and 166 nM insulin for 2 days (day 0 to day 2). This medium was replaced with DMEM containing 10% (v/v) myoclone-plus FCS and 166 nM insulin for a further 2 days (day 2 to day 4). From day 4 onwards cells were maintained in DMEM containing 10% (v/v) myoclone-plus FCS, replacing the medium every two days. 3T3-L1 adipocytes were used between day 7 and day 10 after initiation of differentiation.

Differentiated 3T3-L1 adipocytes were microinjected with the IRAP-GFP construct used in Example 1 (pcDNA3).

Microinjection was carried out using an Eppendorf semi-automatic system (Eppendorf 5171 micromanipulator and Eppendorf 5424 pressure injector) and a Zeiss Axiovert 100TV inverted microscope. Micropipettes (0.2 to 0.5 μm external diameter) were pulled from glass borosilicate capillaries (GC120F-10; Clark Biomedical Instruments) using a Sutter Instrument Co. Model P-97 needle puller. Plasmids were microinjected at 20-200 μg/ml in 0.2×TE buffer with an injection time of 0.2-1.0 s and at 60-200 hPa pressure. During the period of microinjection, cells were incubated in growth medium supplemented with 25 mM HEPES pH 7.4 and 2 mM NaHCO₃. After microinjection, the cells were incubated at 37° C. in the appropriate growth medium for 16-24 h to allow expression of protein encoded by the injected plasmid DNA, before being serum-starved for 2 hours prior to stimulation with or without 100 nM insulin for 30 minutes.

Subsequently, cells were fixed in 4% paraformaldehyde and stained with anti-GFP antibodies, as previously described, without prior permeabilisation, in order to visualise GFP exposed on the surface only.

The total level of the IRAP-GFP expression is indicated by the level of GFP fluorescence detected (this corresponds to the second detection method of the first aspect of the invention). The IRAP-GFP exposed at the cell surface is given by the level of staining with the anti-GFP antibodies (and this corresponds to the first detection method of the first aspect of the invention). Microinjected cells expressing IRAP-GFP were imaged by confocal microscopy using a 16× objective. FIG. 3 shows examples of the images that may be obtained by each detection method. The fluorescence intensity due to IRAP-GFP fluorescence and surface IRAP-GFP immunostaining was computed for each individual cell, using Leica Confocal Software, and a background fluorescence figure subtracted. For each cell, the ratio of background corrected surface:total IRAP-GFP was calculated. The results obtained are illustrated in FIG. 4. Each data point represents the red:green fluorescence ratio for one cell. A high ratio of surface:total IRAP-GFP indicates that the cell has undergone a translocation of IRAP-GFP to the plasma membrane. FIG. 4 demonstrates that in the presence of insulin, a significant proportion of cell undergo translocation detectable using this method.

1. A method of determining whether a candidate compound mimics or antagonizes effects of insulin, comprising:
(a) providing a transfected host cell comprising a nucleic acid sequence which encodes a protein construct comprising an IRAP protein moiety and a detectable protein moiety, wherein the detectable protein moiety is fused to a luminal domain of the IRAP protein moiety and further wherein the detectable protein moiety is capable of being assayed by a first detection method capable of detecting the protein moiety when located exofacially but not when located intracellularly and a second detection method capable of detecting the protein moiety both when located exofacially and when located intracellularly;
(b) contacting the transfected host cell with a candidate compound;
(c) measuring the amount of the exofacial protein moiety by the said first detection method and measuring the total amount of exofacial and intracellular detectable protein moiety by the said second detection method; and
(d) comparing the amount of exofacial detectable protein moiety with the total amount of exofacial and intracellular detectable protein moiety to provide a measure of the extent of GLUT4 vesicle translocation.

2. A method according to claim 1, wherein an antibody epitope is present on the detectable protein moiety, said epitope being detectable by the first detection method which is an immunological detection method.

3. A method according to claim 2, wherein the immunological detection method comprises a step in which the antibody epitope is labelled using a fluorescently labelled antibody having a high affinity for the epitope.

4. A method according to claim 2, wherein the immunological detection method comprises a first step in which the antibody epitope is labelled using a first antibody having a high affinity for the epitope and a second step in which the first antibody is detected by a fluorescently labelled second antibody having a high affinity for the first antibody.

5. A method according to any preceding claim, wherein the detectable protein moiety is one which is capable of generating a characteristic emission spectrum detectable by the said second detection method

6. A method according to claim 5, wherein the detectable protein moiety is a fluorescent protein moiety.

7. A method according to claim 6, wherein the fluorescent protein moiety is a green fluorescent protein.

8. A method of determining whether a candidate compound mimics or antagonizes effects of insulin, comprising:
(a) providing a transfected host cell comprising a nucleic acid sequence which encodes a protein construct comprising an IRAP protein moiety, a first detectable protein moiety which is fused to a luminal domain of the IRAP protein moiety and a second detectable protein moiety which is fused to a cytoplasmic domain of the IRAP protein moiety, wherein the first detectable protein moiety is capable of being assayed by a first detection method capable of detecting the first protein moiety when located exofacially but not when located intracellularly and wherein the second detectable protein moiety is capable of being assayed by a second detection method capable of detecting the second protein moiety when located intracellularly;
(b) contacting the transfected host cell with a candidate compound;
(c) measuring the amount of the first protein moiety by the said first detection method representing the amount of the first protein moiety located exofacially and measuring the amount of the second detectable protein moiety by the said second detection method representing the total amount of the second detectable protein moiety; and
(d) comparing the amount of exofacial first detectable protein moiety with the total amount of the second detectable protein moiety to provide a measure of the extent of GLUT4 vesicle translocation.

9. A method according to claim 8, wherein the first detectable protein moiety comprises an antibody epitope, said epitope being detectable by the first detection method which is an immunological detection method.

10. A method according to claim 9, wherein the immunological detection method comprises a step in which the antibody epitope is labelled using a fluorescently labelled antibody having a high affinity for the epitope.

11. A method according to claim 9, wherein the immunological detection method comprises a first step in which the antibody epitope is labelled using a first antibody having a high affinity for the epitope and a second step in which the first antibody is detected by a fluorescently labelled second antibody having a high affinity for the first antibody.

12. A method according to claim 8, 9, 10 or 11, wherein the second detectable protein moiety is one which is capable of generating a characteristic emission spectrum detectable by the said second detection method.

13. A method according to claim 12, wherein the second detectable protein moiety is a fluorescent protein moiety.

14. A method according to claim 13, wherein the fluorescent protein moiety is a green fluorescent protein.

15. A method according to any preceding claim which is a method of identifying an antagonist of insulin, and which further includes a step in which insulin is added before or after the transfected host cell is contacted with the candidate compound.

16. A method according to claim 15, wherein insulin is added after the transfected host cell is contacted with the candidate compound.

17. An insulin antagonist or mimic when identified by the method of any preceding claim.

18. A chimeric IRAP protein comprising an IRAP protein moiety and a detectable protein moiety, wherein the detectable protein moiety is fused to a luminal domain of the IRAP protein moiety and further wherein the detectable protein moiety is capable of being assayed by a first detection method capable of detecting the protein moiety when located exofacially in a cell but not when located intracellularly and a second detection method capable of detecting the protein moiety both when located exofacially in a cell and when located intracellularly.

19. A chimeric IRAP protein comprising (a) an IRAP protein moiety, (b) a first detectable protein moiety which is fused to a luminal domain of the IRAP protein moiety, said first detectable protein moiety being capable of being assayed by a first detection method capable of detecting the protein moiety when located exofacially in a cell but not when located intracellularly, and (c) a second detectable protein moiety which is fused to a cytoplasmic domain of the IRAP protein moiety, said second detectable protein moiety being capable of being assayed by a second detection method capable of detecting the protein moiety when located intracellularly.

20. A DNA molecule which encodes the chimeric IRAP protein of claim 18 or 19.
